(19) United States
(12) Patent Application Publication
(10) Pub. No.: US 2026/0120810 A1
(43) Pub. Date: Apr. 30, 2026

(54) SYNTHESIS AND USE OF BIOMOLECULE TAPE FOR DATA STORAGE

(71) Applicant: Western Digital Technologies, Inc., San Jose, CA (US)

(72) Inventor: Justin P. KINNEY, San Jose, CA (US)

(73) Assignee: Western Digital Technologies, Inc., San Jose, CA (US)

(21) Appl. No.: 18/928,875

(22) Filed: Oct. 28, 2024

(51) Int. Cl. G16B 50/30; C12Q 1/6869; C25B 3/05; C25B 3/07; C25B 3/09; G01N 27/447

(52) U.S. Cl. CPC G16B 50/30; C12Q 1/6869; C25B 3/05; C25B 3/07; C25B 3/09; G01N 27/44717; G01N 27/44791

(57) ABSTRACT

Disclosed herein are methods and systems of encoding sequences of bits on biomolecules, and methods and systems to read such encoded biomolecules. A method of encoding a sequence of bits on a nucleic acid strand may comprise obtaining a decorated starter material, wherein the decorated starter material comprises a plurality of nucleotides of a same type and a plurality of molecular tags, wherein each nucleotide of the plurality of nucleotides includes a respective molecular tag. The method may further comprise creating an encoded nucleic acid strand by removing from the decorated starter material a subset of molecular tags in particular positions of the decorated starter material, the particular positions corresponding to positions of a predetermined bit value within the sequence of bits.

### BACKGROUND

The use of biomolecules, including deoxyribonucleic acid (DNA), ribonucleic acid (RNA), and proteins, to store data has been proposed because of the density, stability, energy-efficiency, and longevity of biomolecules. For example, a human cell has a mass of about 3 picograms and stores around 6.4 gigabytes (GB) of information. The volumetric density of DNA is estimated to be 1,000 times greater than that of flash memory and its energy consumption 10⁸ times less than that of flash memory. In addition, the retention time of DNA can be significantly greater than that of electronic memory. Thus, DNA can store information reliably over time, as can other biomolecules such as RNA and proteins.

Naturally occurring nucleic acids are negatively-charged polyelectrolytes with four monomers that are covalently bonded to form polymer chains. For DNA, the monomers are the nucleotides adenine (A), thymine (T), guanine (G), and cytosine (C). For RNA, the monomers are the nucleotides adenine (A), uracil (U), guanine (G), and cytosine (C). Each nucleotide includes a phosphate group, a sugar (deoxyribose), and a nitrogenous base (adenine, thymine, cytosine, or guanine).

When DNA or RNA is used for data storage, each of the four nucleotides (also sometimes referred to as bases) can encode up to two bits. As one example of an encoding scheme using DNA, the bits “00” could be converted to A, the bits “01” to T, the bits “10” to G, and the bits “11” to C. Using this example encoding scheme, the bit stream 100111001010 would be encoded as the sequence GTCAGG.

In proposed DNA (or RNA) storage systems, after the data has been encoded into a sequence of nucleotides (e.g., as described above), the next step is chemical synthesis, during which custom sequences of DNA (or RNA) are created in accordance with the sequence of monomers representing the bit stream to be stored. This process typically uses automated machines that can build synthetic molecules base by base. These molecules can then be stored and, at some later time, read using conventional sequencing techniques.

As between synthesis and sequencing, the more significant barrier to using DNA, RNA, or other proteins for data storage is synthesis because of the cost and complexity of synthesizing biomolecules having arbitrary sequences of nucleotides.

Therefore, there is a need for improvements.

### SUMMARY

This summary represents non-limiting embodiments of the disclosure.

In some aspects, the techniques described herein relate to a method of encoding a sequence of bits on a nucleic acid strand, the method including: obtaining a decorated starter material, wherein the decorated starter material includes a plurality of nucleotides of a same type and a plurality of molecular tags, wherein each of the plurality of nucleotides includes a respective molecular tag; and creating an encoded nucleic acid strand by removing from the decorated starter material a subset of molecular tags in particular positions of the decorated starter material, the particular positions corresponding to positions of a predetermined bit value within the sequence of bits.

In some aspects, removing from the decorated starter material the subset of molecular tags includes breaking a carbon-oxygen bond or a carbon-carbon bond between the subset of molecular tags and a subset of the plurality of nucleotides to which the subset of molecular tags is attached.

In some aspects, removing from the decorated starter material the subset of molecular tags in the particular positions of the decorated starter material includes: passing the decorated starter material through a nanopore; and the nanopore removing the subset of molecular tags in the particular positions of the decorated starter material in accordance with the sequence of bits.

In some aspects, the nanopore is a first nanopore, and the method further includes: passing the decorated starter material through a second nanopore; and the second nanopore exerting a force on the decorated starter material, the force being in a direction substantially opposite to a translocation direction of the decorated starter material through the first nanopore.

In some aspects, each of the plurality of molecular tags is a methyl group.

In some aspects, the decorated starter material includes one or more of: deoxyribonucleic acid (DNA) or a strand of methylated cytosine.

In some aspects, creating the encoded nucleic acid strand includes: passing the decorated starter material through a nanopore; and, while the particular positions of the decorated starter material are within the nanopore, applying a voltage to remove from the decorated starter material the subset of molecular tags in the particular positions.

In some aspects, the method further includes: creating the decorated starter material.

In some aspects, creating the decorated starter material includes: chemically or enzymatically synthesizing a DNA oligonucleotide sequence, wherein the DNA oligonucleotide sequence contains only cytosine nucleotides; and chemically or enzymatically methylating the DNA oligonucleotide sequence, wherein chemically or enzymatically methylating the DNA oligonucleotide sequence includes adding a methyl group (—CH₃) at a fifth carbon position of a cytosine ring of each of the cytosine nucleotides.

In some aspects, creating the decorated starter material includes: adding the plurality of molecular tags to the plurality of nucleotides of a strand of starter material.

In some aspects, the strand of starter material includes a strand of cytosine nucleotides, and adding the plurality of molecular tags to the plurality of nucleotides of the strand of starter material includes chemically or enzymatically modifying a cytosine residue in the strand of cytosine nucleotides to include a methyl group (—CH₃) at a fifth carbon position of a cytosine ring.

In some aspects, the decorated starter material includes a strand of methylated cytosine, and removing from the decorated starter material the subset of molecular tags in the particular positions of the decorated starter material includes: passing the strand of methylated cytosine through a nanopore; and the nanopore removing methyl groups from the strand of methylated cytosine in accordance with the sequence of bits.

In some aspects, the nanopore removing methyl groups from the strand of methylated cytosine in accordance with the sequence of bits includes: the nanopore removing a first plurality of methyl groups from positions of the strand of methylated cytosine representing a 0 and leaving intact a second plurality of methyl groups from positions of the strand of methylated cytosine representing a 1, or vice versa.

In some aspects, the nanopore removing a first plurality of methyl groups includes control circuitry selectively applying a voltage across the nanopore in accordance with the sequence of bits.

In some aspects, the method further includes: copying the encoded nucleic acid strand.

In some aspects, the techniques described herein relate to a system for encoding a sequence of bits on a nucleic acid strand, the system including: a nanopore; a first electrode situated on a first side of the nanopore; a second electrode situated on a second side of the nanopore; and control circuitry configured to: obtain the sequence of bits, using the first electrode and the second electrode, apply a first voltage across the nanopore in accordance with entries in the sequence of bits that are a first bit value, wherein the first voltage is insufficient to remove a molecular tag from a monomer translocating through the nanopore, and using the first electrode and the second electrode, apply a second voltage across the nanopore in accordance with entries in the sequence of bits that are a second bit value, wherein the second voltage is sufficient to remove the molecular tag from the monomer translocating through the nanopore.

In some aspects, the monomer includes cytosine, and the molecular tag is a methyl group.

In some aspects, the monomer is included in a homopolymer, and wherein the homopolymer is methylated cytosine.

In some aspects, the techniques described herein relate to a system for encoding a sequence of bits on a nucleic acid strand, the system including: a nanopore; and means for applying a voltage across the nanopore in accordance with the sequence of bits, wherein the voltage is sufficient to remove, from a nucleotide of a nucleic acid strand translocating through the nanopore, a molecular tag attached to the nucleotide by breaking a bond between the nucleotide and the molecular tag.

In some aspects, the system further includes: means for exerting a force on the nucleic acid strand, the force being in a direction substantially opposite to a translocation direction of the nucleic acid strand through the nanopore.

In some aspects, the techniques described herein relate to a method of reading a data-storing biomolecule using a nanopore, the data-storing biomolecule storing a first bit value as nucleotides including molecular tags and a second bit value as nucleotides lacking the molecular tags, the method including: detecting an ionic current as the data-storing biomolecule translocates through the nanopore; performing a comparison of the ionic current and a baseline ionic current profile for the nanopore; and based at least in part on the comparison of the ionic current and the baseline ionic current profile for the nanopore, determining a bit pattern stored by the data-storing biomolecule.

In some aspects, the baseline ionic current profile is specific to the nanopore.

In some aspects, the method further includes at least one of: retrieving the baseline ionic current profile from a database; or creating the baseline ionic current profile for the nanopore.

In some aspects, creating the baseline ionic current profile for the nanopore includes at least one of: detecting the ionic current as an undecorated biomolecule translocates through the nanopore, wherein the undecorated biomolecule does not include any molecular tags; or detecting the ionic current as a fully-decorated biomolecule translocates through the nanopore, wherein each nucleotide of the fully-decorated biomolecule includes a respective molecular tag.

In some aspects, the baseline ionic current profile is a first baseline ionic current profile, and the comparison is a first comparison, and further including: performing a second comparison of the ionic current and a second baseline ionic current profile for the nanopore, and wherein determining the bit pattern stored by the data-storing biomolecule is based at least in part on the second comparison.

### BRIEF DESCRIPTION OF THE DRAWINGS

Objects, features, and advantages of the disclosure will be readily apparent from the following description of certain embodiments taken in conjunction with the accompanying drawings in which:

FIG. 1 illustrates methylated cytosine in accordance with some embodiments.

FIG. 2A illustrates a starter material in accordance with some embodiments.

FIG. 2B illustrates a decorated starter material in accordance with some embodiments.

FIG. 2C illustrates an encoded biomolecule in accordance with some embodiments.

FIG. 3A illustrates an example of a system for encoding a sequence of bits on decorated starter material in accordance with some embodiments.

FIG. 3B illustrates an example of a system that includes a speed-control mechanism in accordance with some embodiments.

FIG. 4A is a flow diagram of a method of encoding a sequence of bits on a biomolecule in accordance with some embodiments.

FIG. 4B is a flow diagram of a method that can be used to remove a subset of molecular tags in accordance with some embodiments.

FIG. 5 illustrates a system for reading encoded biomolecules in accordance with some embodiments.

FIG. 6 is an illustration of how read circuitry can compare the detected ionic current to at least one baseline ionic current profile to determine presence and/or absence of molecular tags in accordance with some embodiments.

FIG. 7 is a flow diagram of a method of reading a data-storing biomolecule using a nanopore in accordance with some embodiments.

To facilitate understanding, identical reference numerals have been used, where possible, to designate identical elements that are common to the figures. It is contemplated that elements disclosed in one embodiment may be beneficially utilized in other embodiments without specific recitation. Moreover, the description of an element in the context of one drawing is applicable to other drawings illustrating that element.

### DETAILED DESCRIPTION

The discussion below typically refers to DNA for simplicity, but it is to be appreciated that other biomolecules can be used (e.g., RNA or proteins). It is to be appreciated that the disclosures herein in the context of DNA are applicable to biomolecules in general, including RNA and proteins.

### Synthesis of Data-storing Biomolecules

Data can be stored on (or by) a biomolecule using a synthesis procedure in which a sequence of bits to be stored is somehow mapped to (encoded by) the biomolecule. State-of-the-art DNA synthesis uses electrochemical phosphoramidite chemistry, which integrates electrochemical techniques with phosphoramidite chemistry. Phosphoramidite chemistry is the conventional method for synthesizing oligonucleotides (short DNA sequences) in a controlled, stepwise manner. The process typically begins with a dimethoxytrityl (DMT) group removal in which the 5′-hydroxyl protecting group (DMT) of the nucleotide is removed. Next, a phosphoramidite nucleotide is activated by tetrazole and added to the growing chain, forming a phosphite triester bond. Unreacted hydroxyl groups are then capped to prevent side reactions. The phosphite triester bond is then oxidized to form a stable phosphate backbone. Next, the final oligonucleotide is deprotected and cleaved from the solid support.

In state-of-the-art DNA synthesis, electrochemistry techniques are used to improve certain steps of the phosphoramidite chemistry, such as the oxidation and deprotection steps. For the oxidation step, traditional phosphoramidite chemistry uses iodine and water (or other oxidizing agents) to convert the phosphite triester into a stable phosphate group. This step can be slow, generate chemical waste, and require excess quantities of reagents. In electrochemical methods, the oxidation step is performed electrochemically, and an electric current directly drives the oxidation process at an electrode. This approach can eliminate the need for chemical oxidizing agents, making the process more efficient and more environmentally friendly. In addition, electrochemical oxidation can be controlled more precisely, leading to faster and more uniform conversion, which is helpful for high-throughput oligonucleotide synthesis. For the deprotection step, instead of using strong acids (e.g., trichloroacetic acid), which can be harsh and produce chemical waste, electrochemical methods use an applied potential to selectively break bonds in the protecting groups, which facilitates a cleaner and more selective process.

Relative to conventional phosphoramidite chemistry, electrochemical phosphoramidite chemistry provides several benefits. For example, electrochemical reactions can be precisely controlled via voltage and current, leading to more accurate and reproducible synthesis. In addition, electrochemical phosphoramidite chemistry uses fewer chemical reagents and cleaner reaction conditions, which can lead to fewer side products and impurities, improving the overall quality of the synthesized oligonucleotide. Electrochemical techniques can also be automated and scaled for high-throughput oligonucleotide synthesis, reducing time and cost of the overall process.

Although the use of electrochemical techniques with phosphoramidite chemistry provides some improvements relative to conventional DNA synthesis techniques, there are still some drawbacks. For example, although electrochemical phosphoramidite chemistry reduces the use of chemical reagents and solvents (such as iodine for oxidation or acids for deprotection), these techniques are still relatively wasteful and, therefore, expensive. Because each nucleotide is added in a separate cycle that includes all of the steps (i.e., DMT group removal, nucleotide coupling, capping, oxidation, and deprotection and cleavage), waste is created during each cycle and for each added nucleotide. In addition, the cost of DNA synthesis, even with electrochemical phosphoramidite chemistry, is high, making large-scale DNA storage prohibitively expensive for many applications. Specialized equipment is needed to synthesize custom molecules that store specific sequences as nucleotides (i.e., by mapping bits or bit sequences to specified nucleotides).

Methylation is a biochemical process that involves the addition of a methyl group (—CH₃) to a molecule (e.g., of DNA). Methylation can affect gene expression without changing the DNA sequence. When methylation occurs in the promoter region of a gene, it generally suppresses gene transcription, effectively “turning off” the gene. In naturally-occurring biomolecules, methylation contributes to and partially determines cellular differentiation and development. A characteristic of methylation is that it is stable enough to be inherited through multiple cell divisions without altering the underlying DNA sequence.

In many organisms, including human beings, DNA methylation typically occurs at cytosine bases, especially in the context of CpG dinucleotides (where a cytosine nucleotide is followed by a guanine nucleotide). The methylation occurs at the 5′position of the cytosine ring, forming 5-methylcytosine.

The inventor of the present disclosure had the insight that molecular tags, such as methyl groups, could be taken advantage of to reduce the cost, complexity, and waste of the synthesis process relative to processes that synthesize bespoke biomolecules in which individual bases represent single bits or combinations of bits (e.g., bits “00” represented by A, bits “01” represented by T, bits “10” represented by G, and bits “11” represented by C). Specifically, the inventor had the insight that a single-monomer biomolecule (e.g., a strand that contains only cytosine nucleotides) can be synthesized, chemically or enzymatically, such that each monomer of the biomolecule (which is a homopolymer) has a respective molecular tag (e.g., a methyl group) attached to it. Such a biomolecule can then be used as a data storage “tape.” To write a sequence of bits to the tape, monomers in positions of the biomolecule corresponding to entries that are a predetermined one of the two bit values (either 0 or 1) would have their molecular tags removed in a write process, and monomers in positions of the biomolecule corresponding to entries that are the other of the two bit values (either 1 or 0) would have their molecular tags left in place (intact). To read the stored sequence of bits, the presence or absence of the molecular tags is detected, and the sequence of bits can be reconstructed with knowledge of which bit value is represented by monomers lacking the molecular tags and which bit value is represented by monomers that include the molecular tags.

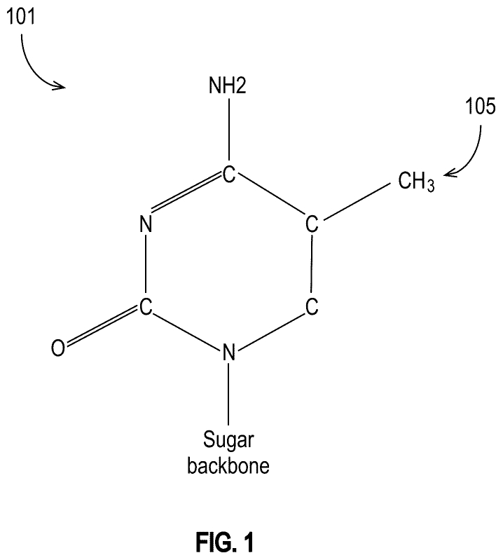

FIG. 1 illustrates a methylated cytosine nucleotide 101 in accordance with some embodiments. As shown, the methylated cytosine nucleotide 101 includes a methyl group 105 on the fifth position of cytosine. Stated another way, a cytosine residue in the methylated cytosine nucleotide 101 has been chemically or enzymatically modified to include the methyl group 105 at the fifth carbon position of the cytosine ring. The methyl group 105 is a type of molecular tag that is attached to (included in) a monomer (cytosine in FIG. 1) in accordance with some embodiments.

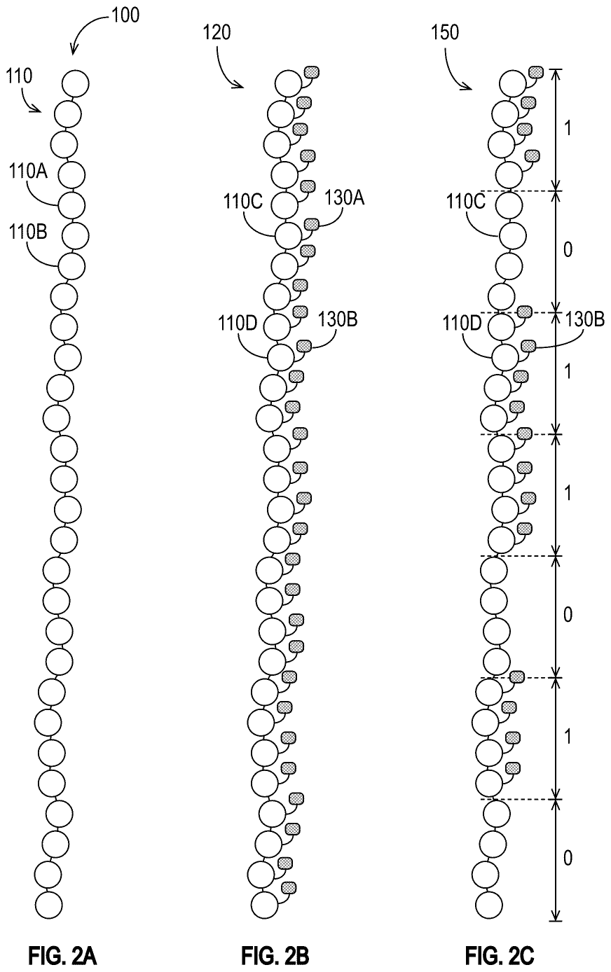

FIGS. 2A, 2B, and 2C illustrate a process of recording bits using a biomolecule in accordance with some embodiments. At a high level, the process involves creating a starter material 100, adding molecular tags 130 to the starter material 100 to create a decorated starter material 120, and then removing subsets of the molecular tags 130 from the decorated starter material 120 to create an encoded biomolecule 150 that stores a sequence of bits.

FIG. 2A illustrates a starter material 100 in accordance with some embodiments. The starter material 100, which can be, for example, a DNA strand, includes individual monomers 110. To avoid complicating the drawing, only two monomers 110 are individually labeled in FIG. 2A, namely the monomer 110A and the monomer 110B. In some embodiments, all of the monomers 110 are of the same type (e.g., a same nucleotide, such as, for example, cytosine). One benefit of all of the monomers 110 being of the same type is that the starter material 100 can be synthesized using a simpler, less expensive process that creates less waste than a process that synthesizes multiple types of monomers into a biomolecule (e.g., two or more of C, T, A, and G).

FIG. 2B illustrates a decorated starter material 120 in accordance with some embodiments. The decorated starter material 120 is the starter material 100 (see FIG. 2A) with molecular tags 130 added to (included in or attached to) the monomers 110. To avoid complicating the drawing, only two monomers 110 and two molecular tags 130 are labeled in FIG. 2B. Specifically, FIG. 2B shows a monomer 110C with a molecular tag 130A attached to it and a monomer 110D with a molecular tag 130B attached to it. The decorated starter material 120 can be thought of as a biomolecule that stores either all “1” values or all “0” values, depending on how the molecular tags 130 are interpreted. A write process then removes molecular tags 130 corresponding to positions (monomers 110) in the decorated starter material 120 that record the other bit value (i.e., if the presence of a molecular tag 130 is interpreted as a “1” bit value, the removal of the molecular tag 130 writes/stores a “0” bit value, and if the presence of a molecular tag 130 is interpreted as a “0” bit value, the removal of the molecular tag 130 writes/stores a “1”bit value).

FIG. 2C illustrates an encoded biomolecule 150 in accordance with some embodiments. It will be appreciated that each individual bit of the sequence of bits being recorded can be represented by multiple consecutive monomers 110. In general, each of the individual bits can be represented by N monomers 110, where N is any positive integer. Representing each bit of a bit stream by multiple monomers 110 can be advantageous to improve the signal-to-noise ratio of the reading (sequencing) process by providing redundancy. In the example illustrated in FIG. 2C, the value of N is 4, and each bit “1” of the bit sequence being recorded is represented by four consecutive monomers 110 with molecular tags 130 attached to them, and each bit “0” of the bit stream is represented by four consecutive monomers 110 without molecular tags 130. Because the monomer 110C is one of four monomers 110 storing a bit value of “0,” the molecular tag 130A is absent in the encoded biomolecule 150. In contrast, because the monomer 110D is one of four monomers 110 storing a bit value of “1,” the molecular tag 130B is present. Reading the stored bits from the bottom of the page to the top of the page, the encoded biomolecule 150 shown in FIG. 2C stores the bit sequence 0101101.

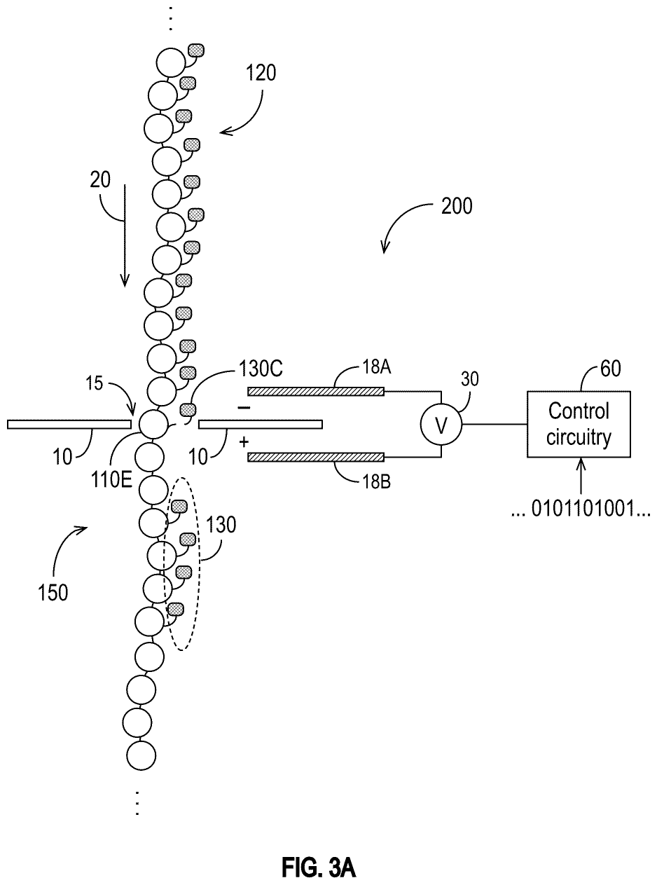

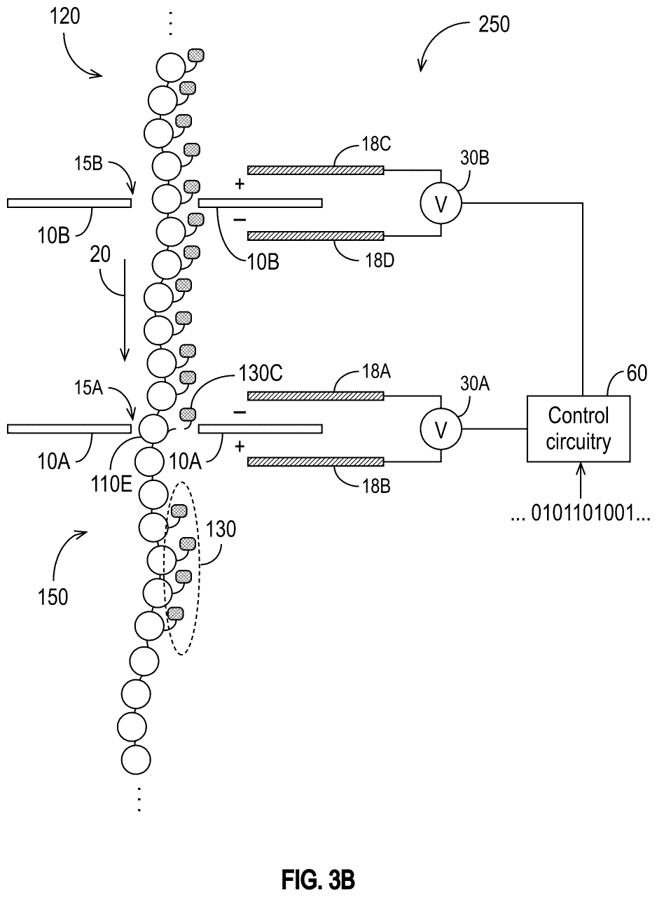

FIG. 3A illustrates an example of a system 200 for encoding a sequence of bits on a biomolecule in accordance with some embodiments. The system 200 is an example of a system that can be used to create the encoded biomolecule 150 from the decorated starter material 120. The system 200 includes a nanopore 15, an electrode 18A, an electrode 18B, a voltage source 30, and control circuitry 60. The decorated starter material 120 enters the nanopore 15, and the encoded biomolecule 150 emerges from the nanopore 15 as a result of the writing process.

The nanopore 15 is a small pore or channel in a membrane 10. The membrane 10 can be a biological material (e.g., a protein) or a synthetic material (e.g., silicon). The diameter of the nanopore 15 (on the order of nanometers) is selected to allow single molecules (e.g., biomolecules) to pass or “translocate” through the nanopore 15. In FIG. 3A, the translocation direction 20 through the nanopore 15 is illustrated by an arrow pointing down.

The electrode 18A and the electrode 18B are coupled to the voltage source 30, which is coupled to and controlled by the control circuitry 60. Negatively-charged biomolecules (e.g., the decorated starter material 120) are drawn into and move through the nanopore 15 at least in part because the voltage source 30, electrode 18A, and electrode 18B cause an electrical potential difference to exist across the nanopore 15 (represented by the “+¿” and “−¿” signs). The electrode 18A and the electrode 18B can be made from any suitable material (e.g., platinum, gold, carbon).

In operation, the control circuitry 60 controls the voltage source 30 to cause the voltage source 30 to selectively apply a time-varying voltage across the nanopore 15 using the electrode 18A and the electrode 18B. (Although not illustrated, the system 200 can also include a reference electrode (e.g., made of Ag or AgCl) to allow the control circuitry 60 to control the applied voltage more precisely.) In particular, the control circuitry 60 controls the voltage source 30 to cause a larger voltage to be applied when needed to break the bond between monomers 110 passing through the nanopore 15 and their respective molecular tags 130. The increased voltage is applied to write whichever of the two bit values (either 0 or 1) is represented by “ordinary” monomers 110 (i.e., monomers 110 without attached molecular tags 130). Thus, as the decorated starter material 120 passes through the nanopore 15, the control circuitry 60 can record/write a particular one of the two bit values by causing the voltage source 30 to apply a larger voltage across the nanopore 15 (using the electrode 18A and the electrode 18B) that is sufficient to break the bond that attaches molecular tags 130 to the monomers 110 representing the particular one of the two bit values. Thus, the control circuitry 60, voltage source 30, and electrode 18A and electrode 18B together apply a voltage across the nanopore 15 in accordance with a sequence of bits, wherein the voltage is sufficient to remove molecular tags 130 from monomers 110 (e.g., by breaking bonds) as they pass through the nanopore 15.

FIG. 3A shows a bit stream portion “01011010010” being provided to the control circuitry 60. In the example of FIG. 3A, each “1” bit is stored as four consecutive monomers 110 with intact molecular tags 130, and each “0” bit is stored as four consecutive monomers 110 without molecular tags 130 (i.e., with the molecular tags 130 removed when the decorated starter material 120 passes through the nanopore 15). In other words, the encoding scheme is the same as shown in FIG. 2C. The control circuitry 60 can keep the molecular tags 130 intact as the decorated starter material 120 passes through the nanopore 15 by controlling the voltage source 30 such that any voltage applied across the nanopore 15 (e.g., to promote electrophoresis) while the monomers 110 in locations of the decorated starter material 120 corresponding to bit values of “1” pass through the nanopore 15 is less than the voltage required to remove the molecular tags 130.

At the moment represented by FIG. 3A, the first and second bits of the bit stream being recorded, a “0” and a “1,” respectively, have already been recorded, and the third bit, a “0,” is in the process of being recorded. The molecular tags 130 have been removed from three of the four monomers 110 that will record/represent the third bit value of “0.” FIG. 3A shows that the bond formerly attaching the molecular tag 130C to the monomer 110E (the third of the four monomers 110 that will represent the third bit value of “0”) has been broken. The control circuitry 60 can continue to apply the higher voltage level across the nanopore 15 while the next monomer 110 passes through the nanopore 15, and then reduce the voltage to the lower level while the next eight monomers 110 (corresponding to the fourth and fifth bit values being “1”) pass through the nanopore 15.

Thus, by controlling the voltage source 30 as the decorated starter material 120 translocates through the nanopore 15, the control circuitry 60 can write a bit stream (a sequence or pattern of bits) to the decorated starter material 120 to produce an encoded biomolecule 150. Specifically, to write whichever of the two bit values is represented by monomers 110 without molecular tags 130, the control circuitry 60 can cause the voltage source 30 to apply across the nanopore 15 (using the electrode 18A and the electrode 18B) a voltage sufficient to break the bond that attaches molecular tags 130 to monomers 110 in order to write that bit value. The control circuitry 60 can write the other bit value by allowing the decorated starter material 120 to pass through the nanopore 15 without an applied voltage (or with an applied voltage that is sufficient to promote electrophoresis but insufficient to break the bonds between the monomers 110 and the molecular tags 130), thereby retaining the molecular tags 130 of the monomers 110 representing the other bit value.

In some embodiments, the decorated starter material 120 comprises methylated cytosine, and the system 200 is configured to perform electrochemical demethylation of the decorated starter material 120. Electrochemical demethylation involves using an electrical current to break the carbon-oxygen or carbon-carbon bond between the methyl group 105 and the molecule (e.g., DNA base or organic compound) to which it is attached (e.g., a cytosine nucleotide). In the example shown in FIG. 3A, the electrode 18A and the electrode 18B, coupled to the voltage source 30, which is controlled by the control circuitry 60, drive the electrochemical reaction. An oxidative or reductive reaction induces the selective removal of the methyl group 105 from specific monomers 110 (e.g., cytosine) as the decorated starter material 120 passes through the nanopore 15. The reaction takes place in a solvent or electrolyte that allows ionic conductivity for the reaction. By applying a positive potential (oxidation), an electron can be removed from the methyl group 105 or the substrate, destabilizing the bond between the methyl group 105 and the rest of the molecule (e.g., a cytosine nucleotide). The bond then breaks and releases the methyl group 105, leaving only regular cytosine. Thus, one way to remove the molecular tag 130C from the monomer 110E is via demethylation.

Demethylation can result in the transformation of 5-methylcytosine into a simple cytosine nucleotide, along with the generation of by-products such as formaldehyde (CH₂O) and protons (H⁺) . These by-products can be allowed to accumulate until their concentration reaches unacceptable levels, at which point the electrolyte bath can be changed. For example, it is undesirable for the formaldehyde to crosslink DNA (i.e., bind to two regions of DNA and form a chemical link), so the electrolyte bath can be changed when the formaldehyde concentration becomes large enough that the probability of crosslinking is considered too high. The protons (H⁺) are reactive and, at high enough concentration, can induce chemical changes to the biomolecule, so the electrolyte bath can be changed when the concentration reaches a specified level. Those having ordinary skill in the art will be able to determine by-product concentrations that should trigger changing of the electrolyte bath.

One challenge presented by the voltage source 30 applying a time-varying voltage across the nanopore 15 to write a sequence of bits to the decorated starter material 120 is that the speed of the decorated starter material 120 through the nanopore 15 is generally proportional to the voltage across the nanopore 15. Thus, the decorated starter material 120 generally moves more slowly through the nanopore 15 at lower voltages and more quickly at higher voltages. Thus, without additional measures in place to control the speed of the decorated starter material 120 through the nanopore 15, the decorated starter material 120 will move more quickly through the nanopore 15 at or around the times when the voltage being applied by the voltage source 30 is at the higher level used to break the bonds between molecular tags 130 and monomers 110.

In some embodiments, the control circuitry 60 takes into account how fast the decorated starter material 120 moves through the nanopore 15 at the two voltage levels (e.g., the lower voltage applied to cause the decorated starter material 120 to be drawn into and move through the nanopore 15 (to leave molecular tags 130 attached to monomers 110), and the higher voltage applied to remove molecular tags 130 from monomers 110) to determine how long to apply each voltage. Because the speed of the decorated starter material 120 may be different depending on which bit value is being written (e.g., if the same number of monomers 110 is used to represent each bit of the bit sequence), the time to write whichever of the bit values is represented by monomers 110 that include molecular tags 130 may be longer than the time to write the bit value that is represented by monomers 110 without molecular tags 130 because of the difference in speed at which the decorated starter material 120 moves through the nanopore 15 with the two applied voltages.

In some embodiments, the number of monomers 110 representing each bit of the sequence of bits being recorded is the same integer number N. For example, in FIGS. 2C and 3A, each bit of the sequence of bits is encoded as (or by) four monomers 110, with each “1” bit being recorded as four monomers 110 with molecular tags 130 and each “0” bit being recorded as four monomers 110 without molecular tags 130. The “0” bit is recorded at the higher voltage level, which means that, absent a speed-control mechanism, the decorated starter material 120 moves through the nanopore 15 more quickly each time a “0” bit is recorded. The control circuitry 60 can take into account how much faster the decorated starter material 120 moves in determining how long to apply each of the voltage levels. For example, if the decorated starter material 120 moves through the nanopore 15 twice as fast at the higher voltage level than at the lower voltage level, time required to record a “0” bit of the bit sequence is half the time required to record a “1” bit. Thus, the control circuitry 60 can cause the voltage source 30 to apply the higher voltage level during a bit-recordation period that is half as long as the bit-recordation period for “1”bits.

In general, in order to write a mix of bit values (a bit stream) to the decorated starter material 120, the control circuitry 60 controls the voltage source 30 to cause the decorated starter material 120 to be subjected to different voltage levels as it passes through the nanopore 15. In some applications, it may be desirable to provide one or more mechanisms to reduce or control the translocation speed of the decorated starter material 120 through the nanopore 15. Such mechanisms can improve the accuracy of the writing process (e.g., to mitigate the effects of latency between when the control circuitry 60 issues a command to the voltage source 30 and when the voltage across the nanopore 15 is modified). A variety of approaches to reduce the speed of the decorated starter material 120 through the nanopore 15 can be implemented.

For example, the speed of the decorated starter material 120 through the nanopore 15 can be reduced by adding certain chemicals or reagents to the electrolyte solution to interact with the decorated starter material 120 and slow its movement. As a specific example, a viscosity-modifying agent (e.g., glycerol) can be added to the solution to increase the resistance experienced by the decorated starter material 120, thereby slowing its translocation through the nanopore 15.

Alternatively, or in addition, the environment of the decorated starter material 120 (e.g., the solution) can be cooled. Lower temperatures decrease the kinetic energy of molecules (such as the decorated starter material 120), making them move more slowly.

As yet another example, specialized enzymes can be attached to the decorated starter material 120 (e.g., DNA helicase). These enzymes help “feed” the decorated starter material 120 through the nanopore 15 at a controlled pace.

The size and shape of the nanopore 15 can also influence the speed at which the decorated starter material 120 passes through it. For example, the nanopore 15 can be made smaller to cause more resistance, which slows the translocation of the decorated starter material 120. The size of the nanopore 15 can be a parameter that is optimized during the design process for the system 200, because the decorated starter material 120 can be defined in advance (e.g., the decorated starter material 120 can be selected as methylated cytosine strands).

Alternatively, or in addition, the hydrodynamic drag exerted on the decorated starter material 120 as it passes through the nanopore 15 can be increased by modifying the fluid dynamics around the nanopore 15. As an example, the flow rates of the fluid on either side of the nanopore 15 can be adjusted to reduce the speed of the decorated starter material 120. As another example, microfluidic devices can be included to manipulate the environment to reduce the speed of the decorated starter material 120.

FIG. 3B illustrates an example of a system 250 that includes a speed-control mechanism in accordance with some embodiments. The system 250 shown in FIG. 3B includes a first nanopore 15A in a first membrane 10A and a second nanopore 15B in a second membrane 10B. The control circuitry 60 is coupled to a first voltage source 30A, which is coupled to an electrode 18A and an electrode 18B, which apply a potential across the first nanopore 15A, and to a second voltage source 30B, which is coupled to an electrode 18C and an electrode 18D, which apply a potential across the second nanopore 15B.

In the example of FIG. 3B, the first nanopore 15A, first voltage source 30A, electrode 18A, and electrode 18B are configured to remove molecular tags 130 from monomers 110 as the decorated starter material 120 passes through the first nanopore 15A. The second nanopore 15B, second voltage source 30B, electrode 18C, and electrode 18D are configured to, in conjunction with the first nanopore 15A, first voltage source 30A, electrode 18A and electrode 18B, control the speed of the decorated starter material 120 as it passes through the first nanopore 15A.

The system 250 is configured to apply differential forces to the decorated starter material 120 to control its speed. In particular, the polarity of the voltage applied by the second voltage source 30B across the second nanopore 15B is opposite the polarity of the voltage applied by the first voltage source 30A across the first nanopore 15A, thereby creating a tug-of-war effect between the forces applied by the first nanopore 15A and the second nanopore 15B. The control circuitry 60 can control the voltages of the first voltage source 30A and the second voltage source 30B such that the speed of the decorated starter material 120 through the first nanopore 15A is a consistent speed, regardless of which of the two bit values is being written to the decorated starter material 120.

For example, assume a voltage of V1 causes the decorated starter material 120 to translocate through the first nanopore 15A at a desired speed, S. Assume further that the first voltage source 30A, electrode 18A, and electrode 18B apply a voltage V2 (where V2>V1) to break the bond between monomers 110 and molecular tags 130, and the voltage V1 otherwise (e.g., to facilitate electrophoresis). To maintain the speed S of the decorated starter material 120 through the first nanopore 15A when molecular tags 130 are being removed from monomers 110, the second voltage source 30B, electrode 18C, and electrode 18D can apply a voltage of V2−V1 across the second nanopore 15B. Because the polarity of the voltage V2−V1 across the second nanopore 15B is opposite the polarity of the voltage V2 applied across the first nanopore 15A, the speed of the decorated starter material 120 through the first nanopore 15A is held at approximately S. When the first voltage source 30A, electrode 18A, and electrode 18B apply the voltage V1 across the first nanopore 15A, the control circuitry 60 can turn off the second voltage source 30B (or set the voltage it supplies to 0), which also results in the speed of the decorated starter material 120 through the first nanopore 15A being approximately S. It is to be appreciated that instead of the second voltage source 30B applying 0 V across the second nanopore 15B when molecular tags 130 are not removed from monomers 110, it may be desirable to use an offset or minimal voltage that is lower than V2, in which case the voltage applied by the first voltage source 30A can be increased by the same offset or minimal voltage.

The second nanopore 15B, second voltage source 30B, electrode 18C, and electrode 18D thus provide one way to exert a force on the decorated starter material 120 as it translocates through the first nanopore 15A, where the force is in a direction substantially opposite to the translocation direction 20.

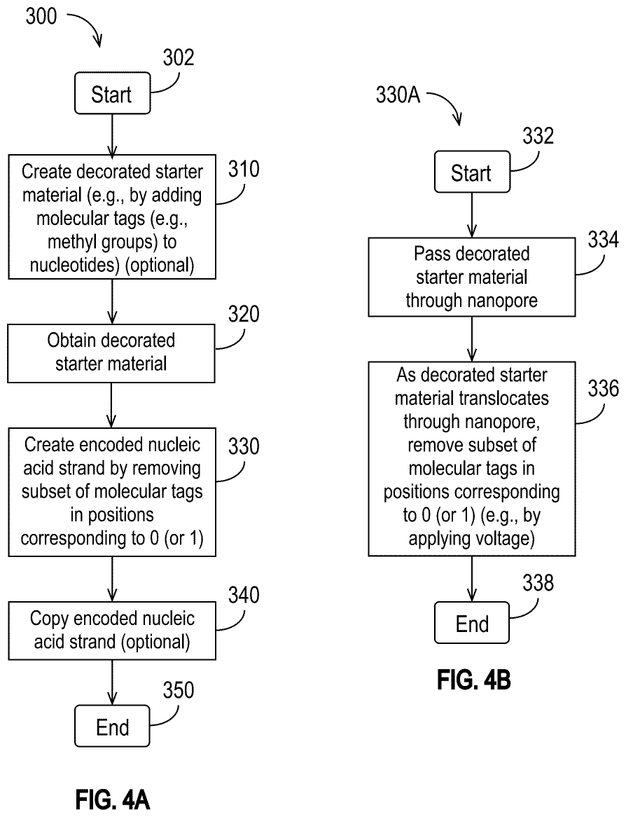

FIG. 4A is a flow diagram of a method 300 of encoding a sequence of bits on a biomolecule (e.g., a nucleic acid strand, such as a strand of cytosine) in accordance with some embodiments. At block 302, the method 300 begins. At block 310, optionally, a decorated starter material 120 is created. The decorated starter material 120 can be created as described above, such as by adding molecular tags 130 to the monomers 110 of a starter material 100. As a specific example, the decorated starter material 120 can be created by methylation of a suitable starter material 100 (e.g., a biomolecule with only cytosine nucleotides as the monomers 110). The decorated starter material 120 can be produced in quantity and provided to entities that wish to record bit sequences. In some embodiments, the decorated starter material 120 is methylated cytosine.

It will be appreciated that block 310 of the method 300 is optional because it may be possible for the entity performing the remaining steps of the method 300 to obtain the decorated starter material 120 from another entity. For example, there may be entities that specialize in creating biomolecules, such as the decorated starter material 120, and these entities may be different from the entities who use the decorated starter material 120 to store bits.

At block 320, a decorated starter material 120 (e.g., methylated cytosine) is obtained. For example, the decorated starter material 120 can be retrieved from local physical storage. Alternatively, if block 310 is performed, the decorated starter material 120 can be obtained as the output of the process(es) performed in block 310.

At block 330, an encoded biomolecule 150 is created by removing a subset of the molecular tags 130 from the decorated starter material 120. Block 330 can be carried out, for example, using the system 200 or the system 250. As explained above, the locations of the removed molecular tags 130 will depend on the selected encoding scheme and the sequence of bits being recorded. For example, if the encoding scheme establishes that the bit value “0” is represented by one or more monomers 110 with no molecular tag(s) 130, and each bit of a sequence being recorded is “written to” an integer number N of monomers 110, then each “0” of the bit stream will be recorded by removing N molecular tags 130 from N monomers 110 of the decorated starter material 120. The positions of the N monomers 110 with molecular tags 130 removed will correspond to the positions of the “0” bits in the sequence of bits being recorded. For example, if the bit being recorded is the first bit of the bit sequence, the molecular tags 130 of the first N monomers 110 available for recording will record the first bit, the next N monomers 110 will record the second bit, etc.

The molecular tags 130 can be removed in any suitable manner. For example, as described above, the molecular tags 130 can be removed using demethylation (e.g., electrochemical demethylation). FIG. 4B is a flow diagram of a method 330A that can be used to perform block 330 of FIG. 4A. The method 330A can be performed, for example, by a system such as the system 200 shown in FIG. 3A or the system 250 shown in FIG. 3B.

With reference to FIG. 4B, at block 332, the method 330A begins. At block 334, the decorated starter material 120 is passed through a nanopore 15. At block 336, as the decorated starter material 120 translocates through the nanopore 15, a subset of molecular tags 130 is removed from monomers 110 in positions corresponding to whichever of the bit values (0 or 1) is represented by monomers 110 without molecular tags 130. For example, if the decorated starter material 120 is a strand of methylated cytosine, the bit value “1” is represented by ordinary cytosine, and the bit value “0” is represented by methylated cytosine, then at block 336, the methyl group 105 at each cytosine nucleotide 101 in a position corresponding to (representing) a “1” is removed. As explained above, the removal can be effected by applying a voltage across the nanopore 15, where the voltage is sufficient to break the bond between cytosine nucleotides 101 passing through the nanopore 15 and their respective methyl groups 105. For example, referring to FIG. 3A, the control circuitry 60 can cause the voltage source 30 to apply an appropriate voltage across the nanopore 15 using the electrode 18A and the electrode 18B.

Referring back to FIG. 4A, after the encoded biomolecule 150 has been created in block 330, at block 340, optionally, the encoded biomolecule 150 is copied. Performing block 340 can be advantageous to provide redundancy and allow the stored sequence of bits to be retained and later read accurately even if the encoded biomolecule 150 or some copies of it are degraded, fragmented, etc. The availability of copies of the encoded biomolecule 150 can also improve the signal-to-noise ratio of the reading process (e.g., multiple versions of the encoded biomolecule 150 can be read, and a majority result provided as the overall read result).

The system 200 shown in FIG. 3A, the system 250 shown in FIG. 3B, and the processes and methods described in the context of FIGS. 2A, 2B, 2C, 3A, 3B, 4A, and 4B can offer a number of potential advantages relative to conventional or other possible approaches to storing data on (as) biomolecules. For example, the process to synthesize methylated cytosine, which, as explained above, is a suitable decorated starter material 120, is simpler than the process to synthesize a bespoke molecule with an arbitrary sequence of nucleotides representing a bit pattern. Chemical methylation can chemically transfer methyl groups to cytosine bases using reagents such as methyl iodide or diazomethane. Because the molecule has no bases other than cytosine, there is no need to protect other bases before methylating the cytosines. After the reaction, purification methods can be used to isolate the methylated cytosine from the reaction mixture.

Another advantage of the writing process is that the use of certain harsh chemicals typically required for demethylation can be avoided by using electrochemical methods. In addition, the reaction can be finely controlled by adjusting the applied voltage, allowing for selective removal of methyl groups 105 to record a bit stream to a biomolecule. Another advantage is that electrochemical methods can potentially be scaled for large-scale or high-throughput synthesis, because the “tape” is always the same regardless of the bit sequence to be recorded, whereas approaches that use nucleotide identities to represent bits require synthesis of custom biomolecules for each bit sequence recorded, which is much harder to scale (e.g., due to cost and complexity).

An additional benefit of the approach disclosed herein is that the typical drawbacks of demethylation are attenuated. For example, achieving selectivity for the methyl group without damaging the underlying DNA structure can be challenging, but because the monomers 110 of the decorated starter material 120 are all the same (e.g., cytosine), there can be some amount of damage, as long as the encoded biomolecule 150 can still be read. In other words, what matters is whether each of the monomers 110 does or does not have a molecular tag 130 attached, not whether the monomers 110 themselves are undamaged or “good.” Similarly, unintended side reactions that lead to the production of reactive oxygen species (ROS) or other by-products that could damage the biomolecule are of less concern here than when the identities of individual nucleotides are important. As long as the presence and absence of the molecular tags 130 can be detected, the encoded biomolecule 150 can be read. Thus, some amount of damage to the biomolecule can be tolerated, and the disclosed techniques offer robustness.

In addition, in typical applications involving demethylation, it is generally desirable to optimize the electrochemical parameters, such as the applied voltage, materials of the electrode 18A and the electrode 18B, and electrolyte composition, for each specific molecule being demethylated. With the disclosed techniques, this optimization can be performed at a single time (e.g., when the system 200 is designed), because the monomers 110 of the decorated starter material 120 are all the same (e.g., cytosine nucleotides), as are the molecular tags 130 (e.g., methyl groups 105).

Another significant advantage of the disclosed techniques is that they are safer than data storage approaches that require bespoke biomolecules to be synthesized to represent sequences of bits. In conventional approaches in which the identity of each nucleotide (e.g., A, T, C, G) represents the bit values, it is possible to inadvertently create a hazardous biomolecule (e.g., a dangerous virus) in order to store a particular sequence of bits. Furthermore, it might not be known ahead of time that the biomolecule being created is potentially dangerous. But the techniques disclosed herein have no such risk. In some embodiments, the starter material 100 and the decorated starter material 120 include only one type of monomer 110 (e.g., cytosine), which does not occur naturally and is non-hazardous, whether fully or partially methylated or demethylated.

### Sequencing of Data-storing Biomolecules

After a sequence of bits has been recorded using the synthesis techniques described above, the encoded biomolecule 150 can be read using any suitable technique. Suitable techniques include, for example, sequencing-by-synthesis (SBS) and nanopore sequencing.

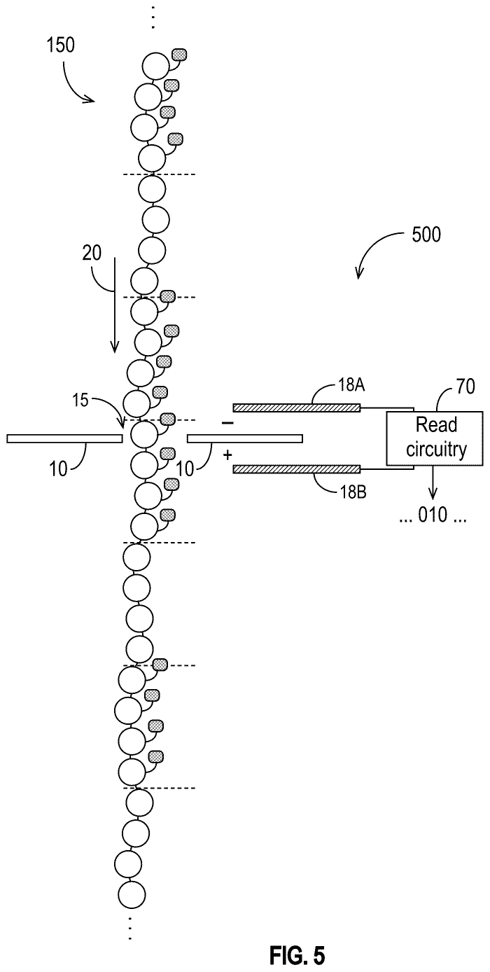

In nanopore sequencing, a configuration similar to the ones shown in FIG. 3A or FIG. 3B can be used. FIG. 5 illustrates a system 500 for reading encoded biomolecules in accordance with some embodiments. The system 500 includes a nanopore 15 in a membrane 10, an electrode 18A, an electrode 18B, and read circuitry 70 coupled to the electrode 18A and the electrode 18B. The nanopore 15, membrane 10, electrode 18A, and electrode 18B can be as described above.

In operation, the encoded biomolecule 150 in an electrolyte solution can be driven through the nanopore 15. The read circuitry 70 causes a highly-focused external electric field to be applied transverse to and in the vicinity of the nanopore 15 (e.g., by the electrode 18A and the electrode 18B). This electric field acts on a relatively short segment of the encoded biomolecule 150 and directs it through the nanopore 15. As the encoded biomolecule 150 passes through the nanopore 15, ions occupying the hole are excluded, which causes changes in the ionic current and/or electronic signal measured across the nanopore 15 (e.g., using the electrode 18A and the electrode 18B). The read circuitry 70 can record the current blockades (e.g., using a current amplifier) and convert them into digital signals (e.g., using an analog-to-digital converter). Thus, the read circuitry 70 can provide, as output, the recovered or read sequence of bits. At the moment represented by FIG. 5, the first three bits of the sequence of bits have been read as “010,” and the fourth bit, a “1,” is in the process of being read.

The read circuitry 70 can include hardware for applying the electric field across the nanopore 15 and for sensing the ionic current through the nanopore 15. Any suitable hardware can be included in the read circuitry 70, such as, for example, one or more voltage sources, one or more current sources, one or more amplifiers (e.g., current amplifiers), one or more digitizers (e.g., analog-to-digital converters), one or more processors, memory, etc.

The system 500 can include any of the translocation speed control mechanisms described above in the context of FIGS. 3A and 3B. Some or all of these mechanisms can be included so that the translocation speed of the encoded biomolecule 150 through the nanopore 15 is substantially consistent.

Methylation of individual or groups of monomers 110 can be inferred from the ionic current through the nanopore 15. In other words, the current blockades, or patterns of them, can be used to distinguish between monomers 110 that include molecular tags 130 and monomers 110 that do not. Thus, nanopore sequencing can detect epigenetic modifications directly by identifying changes in the current disruption caused by modified monomers 110, such as 5-methylcytosine.

In some embodiments, the ionic current is detected as the encoded biomolecule 150 passes through the nanopore 15, and the detected ionic current is compared to at least one baseline ionic current profile. The at least one baseline ionic current profile can include, for example, the ionic current that is detected through the nanopore 15 when an undecorated biomolecule (e.g., the starter material 100) passes through the nanopore 15. Alternatively, or in addition, the at least one baseline ionic current profile can include the ionic current that is detected when a fully decorated biomolecule (e.g., the decorated starter material 120) passes through the nanopore 15. Based at least in part on the comparison between the detected ionic current and at least one baseline ionic current profile, it can be determined which of the monomers 110 of the encoded biomolecule 150 included molecular tags 130 and which did not. For example, if a first baseline ionic current profile represents the ionic current when an undecorated biomolecule (e.g., the starter material 100) translocates through the nanopore 15, the positions of monomers 110 with attached molecular tags 130 can be detected as deviations from the first baseline ionic current profile. Alternatively, or in addition, if a second baseline ionic current profile represents the ionic current when a fully-decorated biomolecule (e.g., the decorated starter material 120) translocates through the nanopore 15, the positions of monomers 110 without attached molecular tags 130 can be detected as deviations from the second baseline ionic current profile. The recorded sequence of bits can then be determined using the value of N (the number of monomers 110 encoding each bit of the sequence of bits).

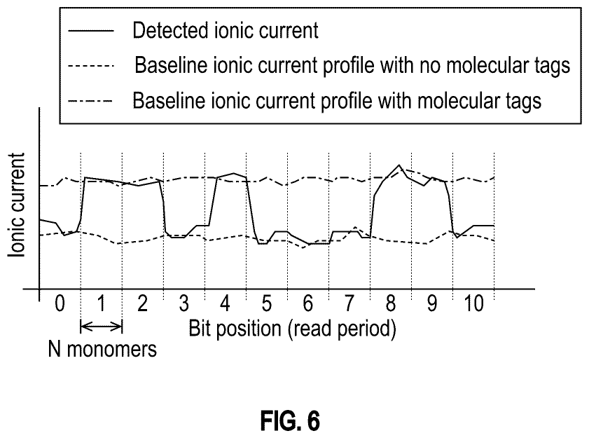

FIG. 6 is an illustration of how the read circuitry 70 can compare the detected ionic current to at least one baseline ionic current profile to determine which of the starter material 100 have molecular tags 130 and which do not. Although FIG. 6 shows both the baseline ionic current profile for a fully-decorated biomolecule (e.g., the decorated starter material 120) and the baseline ionic current profile for an undecorated biomolecule (e.g., the starter material 100), it is to be appreciated that an implementation of the read circuitry 70 can use a single baseline ionic current profile. In the illustrated example, the detected ionic current more closely matches the baseline ionic current profile without molecular tags during the bit periods 0, 3, 5, 6, 7, and 10, and it more closely matches the baseline ionic current profile with molecular tags during the bit periods 1, 2, 4, 8, and 9. Thus, the read circuitry 70 can conclude that the values of the bits in positions 0, 3, 5, 6, 7, and 10 are whichever bit value is represented by monomers 110 without molecular tags 130 (either 0 or 1), and the values of the bits in positions 1, 2, 4, 8, and 9 are the bit value that is represented by monomers 110 with molecular tags 130 (either 1 or 0). If the bit value of 0 is represented by monomers 110 without molecular tags 130, the read circuitry 70 can conclude that the sequence of bits just read, in order of lowest-to-highest-numbered bit position is 01101000110.

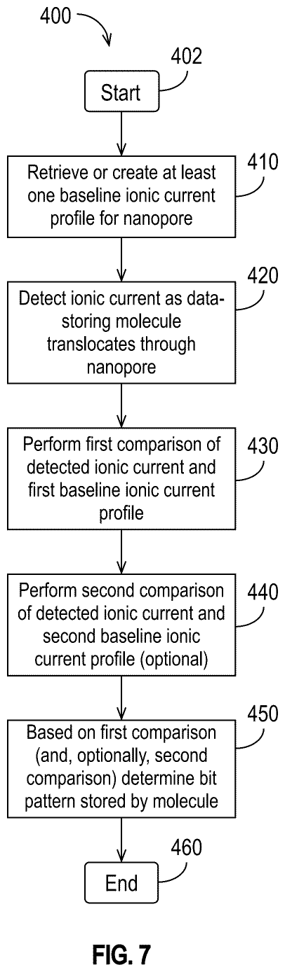

FIG. 7 is a flow diagram of a method 400 of reading a data-storing biomolecule using a nanopore in accordance with some embodiments. The method 400 can be performed, for example, by the system 500 shown in FIG. 5. The biomolecule stores one of the bit values (either 0 or 1) using monomers 110 with attached molecular tags 130 and the other bit value (either 1 or 0) using monomers 110 without any molecular tags 130 attached. At block 402, the method 400 begins. At block 410, at least one baseline ionic current profile for the nanopore 15 being used to read the biomolecule is retrieved or created. The at least one baseline ionic current profile can be specific to the nanopore 15 being used (e.g., determined using measurements from, a model of, or a calibration process performed using the particular nanopore 15 that is being used), or it can be a representative baseline ionic current profile derived from measurements or modeling of multiple nanopores (e.g., similar or identical to the nanopore 15 being used to read the biomolecule). The at least one baseline ionic current profile can be created on the fly (e.g., before reading the biomolecule), or the at least one baseline ionic current profile can be created earlier in time (e.g., during a calibration/qualification procedure performed previously for the nanopore 15) and stored in and retrieved from memory (e.g., a database).

At block 420, the ionic current is detected (e.g., by the read circuitry 70 of the system 500) as the encoded biomolecule 150 passes through the nanopore 15. The current can be detected (e.g., using a current amplifier) and converted into a digital signal (e.g., using an analog-to-digital converter).

At block 430, a first comparison of the detected ionic current and a first baseline ionic current profile is made. As explained above, the first baseline ionic current profile can represent the ionic current when an undecorated biomolecule (e.g., the starter material 100) translocates through the nanopore 15, or it can represent the ionic current when a fully-decorated biomolecule (e.g., the decorated starter material 120) translocates through the nanopore 15.

At block 440, optionally, a second comparison of the detected ionic current and a second baseline ionic current profile is made. If, in block 430, the first comparison was between the detected ionic current and the ionic current when an undecorated biomolecule (e.g., the starter material 100) translocates through the nanopore 15, then, at block 440, the second comparison is between the detected ionic current and the ionic current when a fully-decorated biomolecule (e.g., the decorated starter material 120) translocates through the nanopore 15. Conversely, if, in block 430, the first comparison was between the detected ionic current and the ionic current when a fully-decorated biomolecule (e.g., the decorated starter material 120) translocates through the nanopore 15, then, at block 440, the second comparison is between the detected ionic current and the ionic current when an undecorated biomolecule (e.g., the starter material 100) translocates through the nanopore 15.

At block 450, based on the first comparison (and, optionally, the second comparison, if available), the bit pattern stored by the encoded biomolecule 150 is determined (e.g., by a processor of the read circuitry 70 in the system 500). As explained above, a comparison between the detected ionic current and a baseline current representing the ionic current when an undecorated biomolecule (e.g., the starter material 100) translocates through the nanopore 15 allows the positions of the monomers 110 with attached molecular tags 130 to be identified. Similarly, a comparison between the detected ionic current and a baseline current representing the ionic current when a fully-decorated biomolecule (e.g., the decorated starter material 120) translocates through the nanopore 15 allows the positions of the monomers 110 without attached molecular tags 130 to be identified.

At block 460, the method 400 ends.

In the foregoing description and in the accompanying drawings, specific terminology has been set forth to provide a thorough understanding of the disclosed embodiments. In some instances, the terminology or drawings may imply specific details that are not required to practice the invention.

To avoid obscuring the present disclosure unnecessarily, well-known components are shown in block diagram form and/or are not discussed in detail or, in some cases, at all.

Unless otherwise specifically defined herein, all terms are to be given their broadest possible interpretation, including meanings implied from the specification and drawings and meanings understood by those skilled in the art and/or as defined in dictionaries, treatises, etc. As set forth explicitly herein, some terms may not comport with their ordinary or customary meanings.

As used in the specification and the appended claims, the singular forms “a,” “an” and “the” do not exclude plural referents unless otherwise specified. The word “or” is to be interpreted as inclusive unless otherwise specified. Thus, the phrase “A or B” is to be interpreted as meaning all of the following: “both A and B,” “A but not B,” and “B but not A.” Any use of “and/or” herein does not mean that the word “or” alone connotes exclusivity.

As used in the specification and the appended claims, phrases of the form “at least one of A, B, and C,” “at least one of A, B, or C,” “one or more of A, B, or C,” and “one or more of A, B, and C” are interchangeable, and each encompasses all of the following meanings: “A only,” “B only,” “C only,” “A and B but not C,” “A and C but not B,” “B and C but not A,” and “all of A, B, and C.”

To the extent that the terms “include(s),” “having,” “has,” “with,” and variants thereof are used in the detailed description or the claims, such terms are intended to be inclusive in a manner similar to the term “comprising,” i.e., meaning “including but not limited to.”

The terms “exemplary” and “embodiment” are used to express examples, not preferences or requirements.

The term “coupled” is used herein to express a direct connection/attachment as well as a connection/attachment through one or more intervening elements or structures.

The terms “over,” “under,” “between,” and “on” are used herein refer to a relative position of one feature with respect to other features. For example, one feature disposed “over” or “under” another feature may be directly in contact with the other feature or may have intervening material. Moreover, one feature disposed “between” two features may be directly in contact with the two features or may have one or more intervening features or materials. In contrast, a first feature “on” a second feature is in contact with that second feature.

The term “substantially” is used to describe a structure, configuration, dimension, etc. that is largely or nearly as stated, but, due to manufacturing tolerances and the like, may in practice result in a situation in which the structure, configuration, dimension, etc. is not always or necessarily precisely as stated. For example, describing two lengths as “substantially equal” means that the two lengths are the same for all practical purposes, but they may not (and need not) be precisely equal at sufficiently small scales. As another example, a structure that is “substantially vertical” would be considered to be vertical for all practical purposes, even if it is not precisely at 90 degrees relative to horizontal.

The drawings are not necessarily to scale, and the dimensions, shapes, and sizes of the features may differ substantially from how they are depicted in the drawings.

Although specific embodiments have been disclosed, it will be evident that various modifications and changes may be made thereto without departing from the broader spirit and scope of the disclosure. For example, features or aspects of any of the embodiments may be applied, at least where practicable, in combination with any other of the embodiments or in place of counterpart features or aspects thereof. Accordingly, the specification and drawings are to be regarded in an illustrative rather than a restrictive sense.

1. A method of encoding a sequence of bits on a nucleic acid strand, the method comprising:
obtaining a decorated starter material, wherein the decorated starter material comprises a plurality of nucleotides of a same type and a plurality of molecular tags, wherein each nucleotide of the plurality of nucleotides includes a respective molecular tag; and
creating an encoded nucleic acid strand by removing from the decorated starter material a subset of molecular tags in particular positions of the decorated starter material, the particular positions corresponding to positions of a predetermined bit value within the sequence of bits.

2. The method of claim 1, wherein removing from the decorated starter material the subset of molecular tags comprises breaking a carbon-oxygen bond or a carbon-carbon bond between the subset of molecular tags and a subset of the plurality of nucleotides to which the subset of molecular tags is attached.

3. The method of claim 1, wherein removing from the decorated starter material the subset of molecular tags in the particular positions of the decorated starter material comprises:
passing the decorated starter material through a nanopore; and
the nanopore removing the subset of molecular tags in the particular positions of the decorated starter material in accordance with the sequence of bits.

4. The method of claim 3, wherein the nanopore is a first nanopore, and further comprising:
passing the decorated starter material through a second nanopore; and
the second nanopore exerting a force on the decorated starter material, the force being in a direction substantially opposite to a translocation direction of the decorated starter material through the first nanopore.

5. The method of claim 1, wherein each molecular tag of the plurality of molecular tags is a methyl group.

6. The method of claim 1, wherein the decorated starter material comprises one or more of: deoxyribonucleic acid (DNA) or a strand of methylated cytosine.

7. The method of claim 1, wherein creating the encoded nucleic acid strand comprises:
passing the decorated starter material through a nanopore; and
while the particular positions of the decorated starter material are within the nanopore, applying a voltage to remove from the decorated starter material the subset of molecular tags in the particular positions.

8. The method of claim 1, further comprising:
creating the decorated starter material.

9. The method of claim 8, wherein creating the decorated starter material comprises:
chemically or enzymatically synthesizing a DNA oligonucleotide sequence, wherein the DNA oligonucleotide sequence contains only cytosine nucleotides; and
chemically or enzymatically methylating the DNA oligonucleotide sequence, wherein chemically or enzymatically methylating the DNA oligonucleotide sequence comprises adding a methyl group (—CH₃) at a fifth carbon position of a cytosine ring of each of the cytosine nucleotides.

10. The method of claim 8, wherein creating the decorated starter material comprises:
adding the plurality of molecular tags to the plurality of nucleotides of a strand of starter material.

11. The method of claim 10, wherein:
the strand of starter material comprises a strand of cytosine nucleotides; and
adding the plurality of molecular tags to the plurality of nucleotides of the strand of starter material comprises chemically or enzymatically modifying a cytosine residue in the strand of cytosine nucleotides to include a methyl group (−CH₃) at a fifth carbon position of a cytosine ring.

12. The method of claim 10, wherein:
the decorated starter material comprises a strand of methylated cytosine; and
removing from the decorated starter material the subset of molecular tags in the particular positions of the decorated starter material comprises:
passing the strand of methylated cytosine through a nanopore; and
the nanopore removing methyl groups from the strand of methylated cytosine in accordance with the sequence of bits.

13. The method of claim 12, wherein the nanopore removing methyl groups from the strand of methylated cytosine in accordance with the sequence of bits comprises:
the nanopore removing a first plurality of methyl groups from positions of the strand of methylated cytosine representing a 0 and leaving intact a second plurality of methyl groups from positions of the strand of methylated cytosine representing a 1, or vice versa.

14. The method of claim 13, wherein the nanopore removing a first plurality of methyl groups comprises control circuitry selectively applying a voltage across the nanopore in accordance with the sequence of bits.

15. The method of claim 1, further comprising:
copying the encoded nucleic acid strand.

16. A system for encoding a sequence of bits on a nucleic acid strand, the system comprising:
a nanopore;
a first electrode situated on a first side of the nanopore;
a second electrode situated on a second side of the nanopore; and
control circuitry configured to:
obtain the sequence of bits,
using the first electrode and the second electrode, apply a first voltage across the nanopore in accordance with entries in the sequence of bits that are a first bit value, wherein the first voltage is insufficient to remove a molecular tag from a monomer translocating through the nanopore, and
using the first electrode and the second electrode, apply a second voltage across the nanopore in accordance with entries in the sequence of bits that are a second bit value, wherein the second voltage is sufficient to remove the molecular tag from the monomer translocating through the nanopore.

17. The system recited in claim 16, wherein:
the monomer comprises cytosine; and
the molecular tag is a methyl group.

18. The system recited in claim 16, wherein:
the monomer is included in a homopolymer; and
the homopolymer is methylated cytosine.

19. A system for encoding a sequence of bits on a nucleic acid strand, the system comprising:
a nanopore; and
means for applying a voltage across the nanopore in accordance with the sequence of bits, wherein the voltage is sufficient to remove, from a nucleotide of a nucleic acid strand translocating through the nanopore, a molecular tag attached to the nucleotide by breaking a bond between the nucleotide and the molecular tag.

20. A method of reading a data-storing biomolecule using a nanopore, the data-storing biomolecule storing a first bit value as nucleotides including molecular tags and a second bit value as nucleotides lacking the molecular tags, the method comprising:
detecting an ionic current as the data-storing biomolecule translocates through the nanopore;
performing a comparison of the ionic current and a baseline ionic current profile for the nanopore; and
based at least in part on the comparison of the ionic current and the baseline ionic current profile for the nanopore, determining a bit pattern stored by the data-storing biomolecule.
